(19) United States
(12) Patent Application Publication
(10) Pub. No.: US 2011/0087088 A1
(43) Pub. Date: Apr. 14, 2011

(54) COMPUTER-ASSISTED IDENTIFICATION AND TREATMENT OF AFFECTED ORGAN TISSUE

(75) Inventors: Ronald L. Korn, Paradise Valley, AZ (US); Mark D. Nathan, Lafayette, CA (US); Nabil Dib, Phoenix, AZ (US)

(73) Assignee: CELL GENETICS, LLC, Carlsbad, CA (US)

(21) Appl. No.: 12/614,130

(22) Filed: Nov. 6, 2009

Related U.S. Application Data
(60) Provisional application No. 61/251,210, filed on Oct. 13, 2009.

(51) Int. Cl. A61B 5/055; A61B 5/053

(52) U.S. Cl. 600/411; 600/547

(57) ABSTRACT

Computer-assisted processes are disclosed for using nuclear medicine image studies (e.g. PET or SPECT), preferably in combination with anatomic image studies (e.g., CT or MRI), to identify and quantify regions of affected organ tissue (e.g., myocardial infarcts), and to calculate doses of stem cells or other therapy to deliver to such regions. The resulting (static) image data showing the affected tissue may also be integrated with live (moving) image data, such as a fluoroscopy image, during a subsequent interventional procedure to generate a hybrid image showing the real time location of an injection catheter relative to the affected tissue.

### RELATED APPLICATIONS

This application claims the benefit of U.S. Provisional Appl. No. 61/251,210, filed Oct. 13, 2009, the disclosure of which is hereby incorporated by reference.

This application is being filed concurrently with a non-provisional patent application titled MEDICAL IMAGING PROCESSES FOR FACILITATING CATHETER-BASED DELIVERY OF THERAPY TO AFFECTED ORGAN TISSUE, which contains substantially the same disclosure as the present application and which claims priority to the provisional application referenced above.

### BACKGROUND OF THE INVENTION

1. Field of the Invention

This disclosure relates to medical imaging technologies and procedures for identifying and quantifying myocardial infarcts and/or other areas of affected organ tissue, and for delivering stem cell therapy, gene therapy, protein therapy, pharmaceutical therapy, device therapy, and/or other types of therapy to the affected tissue.

2. Description of the Related Art

A myocardial infarct or scar is a localized area of dead or damaged myocardial tissue resulting from a heart attack. A myocardial infarct may be treated by injecting an appropriate therapeutic substance, such as stem cells or a pharmaceutical compound, into the damaged tissue using an injection catheter.

A known procedure for identifying and treating myocardial infarcts involves the use of the NOGA™ Cardiac Navigation system to generate a three dimensional (3D) map of the heart. The physician initially uses a special catheter system to generate measurements of electrical activity (voltage) along the inner surface (endocardium) of the left ventricle. These measurements are combined with catheter-tip location data (generated using position sensors) to generate the map. The physician then uses this map (typically during the same catheterization procedure) to select injection locations for delivering stem cells and/or other therapy to the damaged myocardial tissue.

One problem with the above approach is that a high degree of skill is required to take the measurements needed to generate the 3D map. Another problem is that the map, even if generated by a highly skilled physician, does not accurately reveal the mass of the scar tissue, and thus does not provide sufficient information for determining the amount of therapy to deliver. Yet another problem is that the physician ordinarily must devote a significant amount of time (typically 45 minutes or more) to generating the map.

Similar issues exist in connection with the identification and treatment of other types of damaged or otherwise affected cardiac issue (e.g., peri-infarct tissue), and with the identification and treatment of affected tissue of other organs (e.g., the kidneys, brain, liver, bladder, spleen, and pancreas). In general, existing medical imaging technologies and procedures often do not enable physicians to determine the precise locations and boundaries of the affected organ tissue, or to accurately calculate the volume or mass of such tissue. Without such information, the physician typically cannot accurately administer therapy, such as stem cell, gene, pharmaceutical, protein, and/or device therapy. Existing imaging technologies used for catheterization procedures generally do not provide sufficient information for enabling physicians to accurately and reliably deliver therapy to areas of interest.

Nothing in this background section is intended to define or limit the scope of protection.

### BRIEF DESCRIPTION OF THE DRAWINGS

FIG. 1 illustrates a process for identifying and treating damaged or abnormal cardiac tissue (or other organ tissue) according to one embodiment.

FIG. 2 illustrates the general flow of information between system components in one embodiment of the process of FIG. 1.

FIG. 3 illustrates processes that may be used to identify, and calculate the mass of, damaged or abnormal cardiac tissue in the embodiment of FIG. 1.

FIG. 4 illustrates the fusion of a nuclear scan image data with an anatomic scan image.

FIG. 5 illustrates a process for identifying and classifying damaged or abnormal organ tissue using nuclear data, and for visually representing such classified tissue in an anatomic or fused anatomic/nuclear image.

FIG. 6 illustrates the division of an image of an organ into angular sectors for analysis.

FIG. 7 illustrates the application of a threshold method to the image data and sectors of FIG. 6.

FIG. 8 shows how the damaged or abnormal tissue identified using nuclear scan data can be visually represented via color coding in an anatomic image.

FIG. 9 illustrates one example of a process for integrating PET/CT image data, or other data obtained from a combination of nuclear and anatomic scans, with live fluoroscopy images.

FIG. 10 illustrates a process for fusing static/non-invasive image data with a live fluoroscopy image.

FIG. 11 further illustrates how static image data showing scar tissue (and/or other damaged or abnormal tissue) can be integrated or fused with a live fluoroscopy image during a catheterization procedure.

### DETAILED DESCRIPTION OF THE PREFERRED EMBODIMENTS

Specific medical imaging technologies and procedures will now be described for identifying, quantifying and treating myocardial infarcts or other damaged or affected organ tissue. Although the following description focuses on detecting and treating damaged tissue of the heart, as will be apparent, aspects of the disclosed methods are also applicable to disorders involving dead or damaged tissue of other organs, such as the kidneys, brain, liver, bladder, spleen, and pancreas.

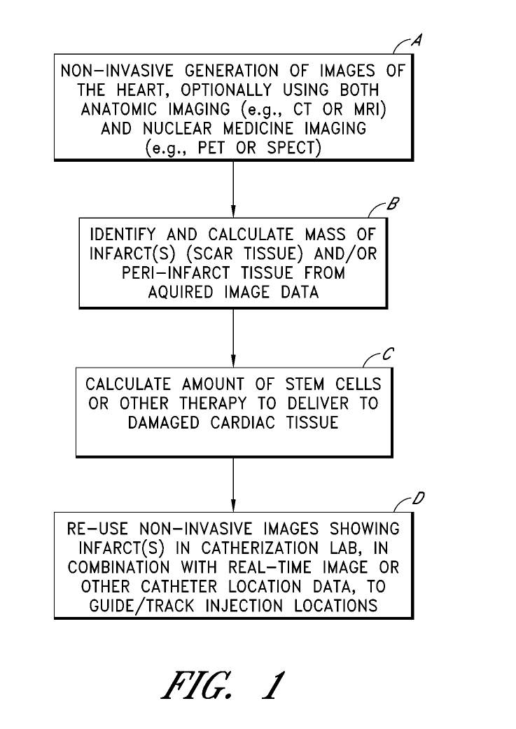

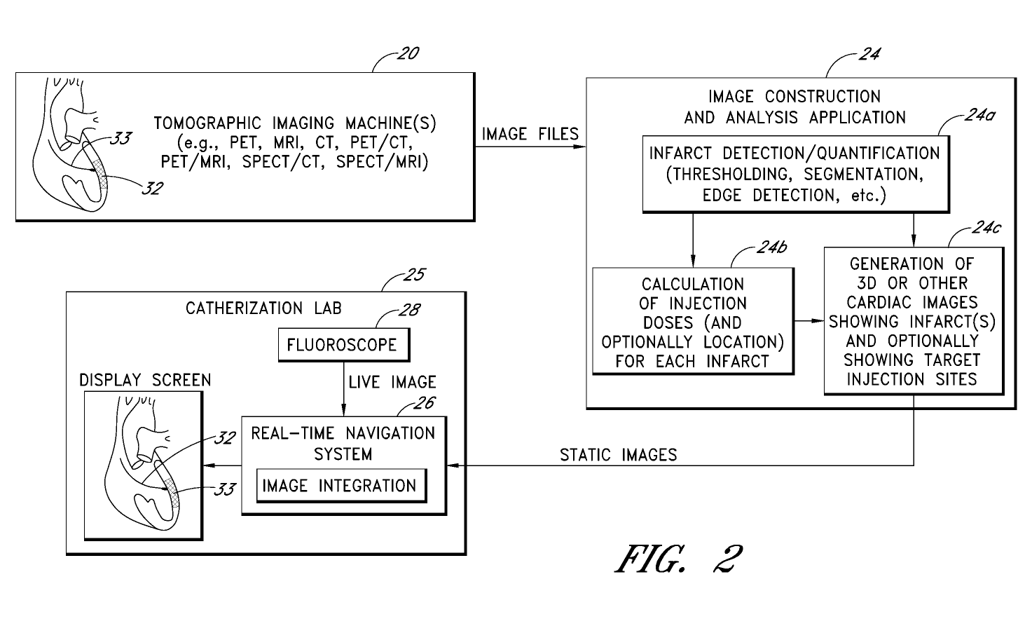

### I. OVERVIEW (FIGS. 1 AND 2)

FIG. 1 illustrates an overall process, depicted as four steps or blocks A through D, for identifying, quantifying and treating one or more myocardial infarcts (also referred to as scar tissue), and/or damaged or ischemic tissue surrounding such infarcts (referred to as “peri-infarct tissue”). Example implementation details of these four steps are described in further detail in subsequent sections. As will be apparent, the process shown in FIG. 1 can also be applied to organs other than the heart.

In step A of FIG. 1, one or more non-invasive imaging technologies/modalities are used to generate scans of the patient's heart. In some embodiments, tomography scans of the heart are generated using both a nuclear medicine imaging process and a non-nuclear/anatomic imaging process. Examples of nuclear medicine imaging processes include positron emission tomography (PET), single photon emission computed tomography (SPECT), and other scanning modalities that use radiotracer techniques. Examples of non-nuclear, anatomic imaging processes include x-ray computerized tomography (CT) and magnetic resonance imaging (MRI). As part of this initial step or phase, a contrast-enhanced CT or MRI scan of the heart may be generated for later fusing or otherwise combining non-invasive image data with fluoroscopy images or other real-time (live) data.

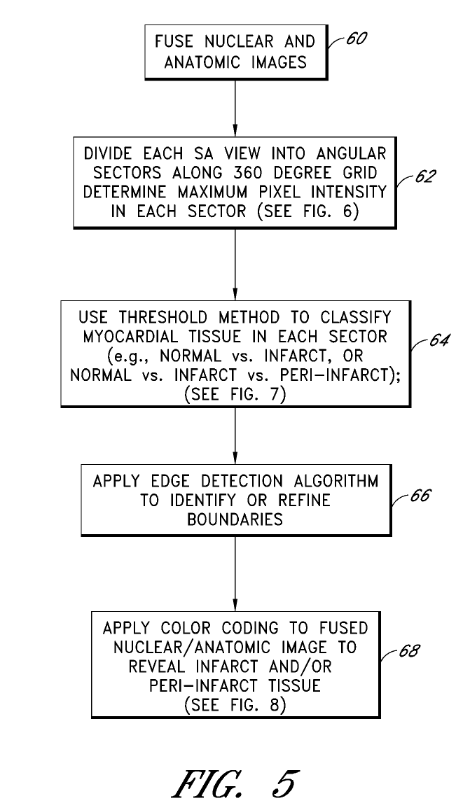

In step B of FIG. 1, the images resulting from step A are used to identify the boundaries, and calculate the mass, of any myocardial infarcts (scar tissue). The boundaries and mass of any peri-infarct regions, and/or other affected regions, may additionally (or alternatively) be identified as part of this process. If both nuclear medicine (e.g., PET or SPECT) and anatomic (e.g., CT or MRI) scans are performed in step A, both types of images are preferably used in combination to calculate the mass of each infarct and/or peri-infarct region. For example, PET or SPECT scans may be used to reliably identify the affected (infarct and/or peri-infarct) tissue, and corresponding CT or MRI scans (which are more reliable for calculating tissue mass) may be used to calculate the mass of such tissue. This may be accomplished in part using well known image fusion methods to combine or fuse corresponding images (e.g., PET with CT, PET with MRI, SPECT with CT, or SPECT with MRI). A specific example of a computerized process for determining infarct and/or peri-infarct boundaries and mass using fused PET and CT images is described below with reference to FIGS. 5-8.

Although the combined use of nuclear medicine scans and anatomic scans provides certain benefits, the infarct (and/or peri-infarct) boundaries and mass may alternatively be calculated based solely on a single cardiac scan, such as a PET scan, a CT scan, or an MRI scan. For example, a contrast enhanced MRI or CT scan can be generated using delayed hyper enhancement (with a delay of 2 to 20 minutes) to identify any myocardial infarcts. The resulting images/slices may then be analyzed to identify the boundaries of the myocardial infarcts. The total voxel volume and mass of each infarct may then be calculated using methods similar to those described herein.

In step C of FIG. 1, the mass calculation(s) resulting from step B are used to calculate the quantity of stem cells and/or other therapy (e.g., gene, protein, or pharmaceutical therapy) to deliver to the affected tissue. A separate calculation may be performed for each identified infarct (or other region of affected tissue), and the result of each such calculation may be used to determine the number of injections to be made into the affected tissue and the dose of each such injection. The accuracy of these dose calculations is important to the efficacy of the treatment; for example, if the quantity of stem cells injected into an infarct or peri-infarct region is too large, the therapy can result in further damage to the myocardium or undesirable complications such as cardiac arrhythmias. Because the dose calculations in the preferred embodiment are based on accurate volume and/or mass calculations (preferably generated in-part using anatomic scans), the doses are more likely to be accurate than with prior art approaches. The therapy applied to the affected areas may be directed to regeneration of muscle, blood vessels, or both.

Steps B and C of FIG. 1 are preferably partially or wholly automated via software executed by one or more machines. For example, image processing software may automatically detect the infarct and/or peri-infarct boundaries in each image or slice, and may also perform the associated calculations for determining the mass of the affected tissue and the doses of the associated injections. The image processing software may also provide an appropriate user interface that enables a physician to verify or control the determination of the identified boundaries.

In step D of FIG. 1, some or all of the non-invasive images generated in step A are re-used in the cardiac catheterization laboratory to assist the physician in interactively positioning the tip or other delivery portion of the injection catheter during an interventional procedure. (In the following description, the injection/delivery portion is assumed to be located at the distal end or tip of the injection catheter, although this need not be the case.) More specifically, real time images and/or data reflective of the current location of the injection catheter's tip is fused or otherwise integrated with the non-invasive image data to generate a real time display showing the location of the catheter tip relative to the affected tissue. This may be accomplished in a variety of ways, including the following:

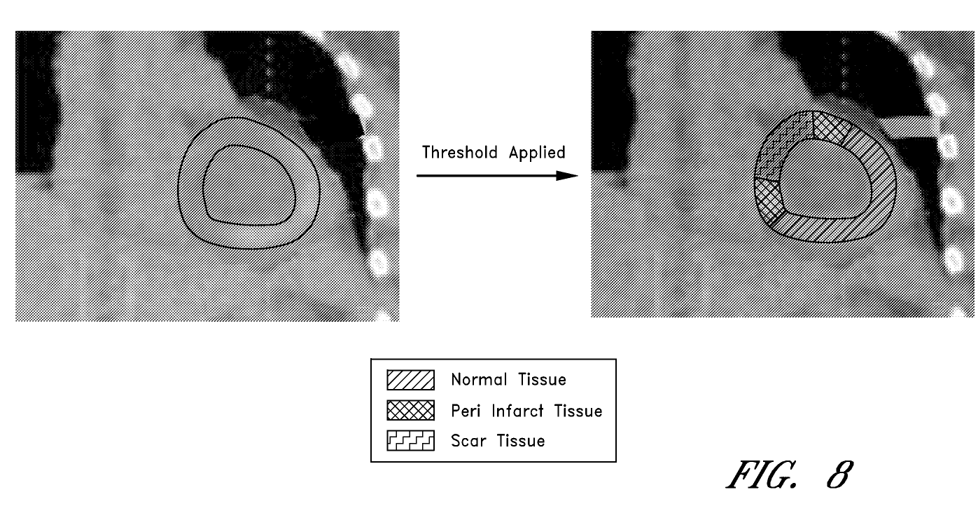

Method 1: A 3D rendering of the heart (or at least the left ventricle) is generated showing the affected tissue (infarct and/or peri-infarct) via color coding. This 3D rendering may be generated based on CT or MRI scans alone, but is more preferably generated using fused PET/CT, PET/MRI, SPECT/CT or SPECT/MRI images. All or a selected portion of this 3D rendering (e.g., the portion showing the affected tissue) is subsequently fused in real time with a fluoroscope-based moving image to effectively superimpose a color-coded representation of the affected tissue onto the fluoroscopy view. One example of how this first method may be performed is described below with reference to FIGS. 8-10.

Method 2: A 3D rendering is generated as in method 1. Real time data regarding the location of the catheter tip is then used to paint or draw a representation of the catheter tip in the 3D rendering. The real time location data may be derived from fluoroscopy images, and/or may be generated using a magnetic, impedance-based, and/or other position sensor located near the tip of the catheter. Examples of sensor-based catheter navigation systems that may be used for this purpose are described in U.S. Pat. No. 7,536,218, the disclosure of which is hereby incorporated by reference. In some embodiments, the physician may be able to rotate the 3D view of the heart via a touch screen or other user interface so that the regions of interest can be viewed from various angles.

The image generated by method 1 or 2 (or another method in which static images are combined with real time data) is referred to herein as a “hybrid image.” The hybrid image, which may include a moving image, is preferably generated via execution of software on a machine during the interventional catheterization procedure.

During the catheterization procedure, the physician may percutaneously insert the injection catheter into a femoral artery, and then advance the catheter tip through the ascending aorta and into the left ventricle. The physician may then use the hybrid image to guide the catheter tip to one or more desired injection locations along the inner wall of the left ventricle. In the case of stem cells, the physician may select multiple injection locations within or around a single infarct, such that the stem cells are appropriately distributed in the region of the scar tissue. To assist with this process, the software that generates the hybrid image may display dots or other visual markers that represent target injection locations. These locations may, in some embodiments, be selected automatically by the software based on infarct size and mass calculations. The software may also generate an audible or other alert when the catheter tip is determined to be in, or within a predefined distance (e.g., a half centimeter) of, a target injection location.

The software that generates the hybrid image may additionally or alternatively update the hybrid image during the catheterization procedure to visually indicate the locations/sites of the actual injections. This feature may be implemented using a special catheter or catheter sensor that detects injection events and reports these events to the software. Alternatively, the software or associated computer may include a user interface (e.g., a physical button or a touchscreen button) that enables the physician to manually indicate that an injection is being performed. In either case, whenever an injection is performed, the software may capture/store information regarding the location of the catheter tip (and injection needle), and visually mark this location in the hybrid image. In some cases, the software may also track, and visually depict in the hybrid image, the volume (dose) of each injection

FIG. 2 illustrates the primary machinery and other components that may be used to carry out the process of FIG. 1. The machinery includes one or more tomographic imaging machines or scanners 20 that are used to generate the non-invasive images in step A of FIG. 1. The machine or machines 20 may, for example, include a PET, MRI, CT, PET/CT, PET/MRI, SPECT/CT or SPECT/MRI scanner. The use of a PET/CT or PET/MRI scanner is particularly useful (but not essential), as such scanners enable the efficient and accurate generation of fused PET/CT or PET/MRI images that are well suited for calculating scar tissue mass.

As further illustrated in FIG. 2, the image files generated by the scanning machinery 20 are passed to an image construction and analysis software application 24. This application 24 may, but need not, run in whole or in part on a computer system (not shown) that is separate from the scanning machinery 20. This computer system may, in some cases, include multiple distinct computers or other machines that interact with each other over a network. The software application 24 may, in some embodiments, include existing application software for analyzing PET, SPECT, and/or other types of imaging studies; one example of a software application that may be used for this purpose is the Emory Cardiac Toolbox available from Syntermed, Inc.

In the embodiment shown in FIG. 2, the software application 24 includes the following software modules or components: an infarct detection/quantification component 24A, a component 24B that calculates injection doses and (optionally) injection locations, and a component 24C that generates the 3D renderings that are used in the catheterization lab. As depicted in FIG. 2, the infarct detection/quantification component may implement various types of algorithms, including a thresholding algorithm and edge detection algorithms for detecting scar tissue boundaries, and a segmentation algorithm for dividing the heart into segments. An example of how such algorithms may be used to automatically identify and quantify scar tissue is described below with reference to FIGS. 5-8. The image construction and analysis application 24 may also include a user interface (not shown) that enables a medical practitioner to perform various functions, such as confirming, modifying, or manually specifying the boundaries of infarcts.

Component 24C in FIG. 2 generates a 3D (or possibly 2D) rendering/view of the heart for use in the catheterization lab 25. As explained above, this rendering shows the identified scar tissue, and is used during the catheterization procedure to navigate the catheter tip to desired injection locations. As depicted by the arrow labeled “static images” in FIG. 2, this rendering (or a selected portion of it) is loaded onto a real time navigation system 26, or some other type of computer system that is used during the catheterization procedure to monitor catheter position. One example of a real time navigation system 26 that may be used is the EP Navigator™ system available from Philips.

In the particular embodiment shown in FIG. 2, the real time navigation system 26 fuses or otherwise integrates a real time (moving) image from an X-ray fluoroscope 28 with the pre-generated static image or images to generate a hybrid image that is displayed on the display screen 30. This hybrid image shows the current location of the catheter 32, including the injection needle at its tip, relative to the scar tissue 33 (shown in cross hashing in FIG. 2). The image of the catheter 32 is generated by the fluoroscope 28 in real time. The fluoroscope 28 may, in some embodiments, be capable of generating a 3D fluoroscopy image of the heart, although 2D fluoroscopy may be used. One example of a fluoroscope capable of generating 3D fluoroscopy images is the Dominion Vi 3D Medical Imaging Scanner available from Imaging3, Inc. As mentioned above, the real time and static images may be fused in-part using a contrast-enhanced CT or MRI scan that shows the major vessels and structures of the heart. Hybrid images may be fused in three-dimensional virtual space in such a way that the images retain proper orientation when manipulated in real time in the catheterization laboratory. Examples of image fusion methods that may be used for this purpose are described in the following references, the disclosures of which are hereby incorporated by reference: U.S. Pat. No. 6,351,513, U.S. Pat. Pub. No. 2006/0239524, and Kriatselis et al., “Integration of CT and fluoroscopy images in the ablative treatment of atrial fibrillation,” MEDICAMUNDI, vol. 52/2, pp. 59-63, 2008.

Rather than displaying the actual fluoroscopy image, the real time navigation system 26 may be designed to analyze this image to determine the location of catheter 32 relative to specific portions of the heart. The real time navigation system may then draw a representation of the catheter (or its tip) in the pre-generated image. Further, the real time navigation system could use position sensor data, ultrasound, and/or another appropriate technology to determine to location of the catheter in the heart, in which case the X-ray fluoroscope 28 may be omitted.

In some embodiments, the injection catheter 32 may include a voltage sensor at its tip (or at another delivery portion of the catheter) to enable the physician to measure electrical activity along the inner all of the left ventricle. This allows the physician to confirm that the catheter tip is in contact with scar tissue prior to making an injection. An optical sensor may alternatively be used, in which case the measurements may reflect the tissue's ability to absorb light. When such a voltage or optical sensor is used, the real time navigation system 26 may visually represent the voltage or optical measurements (e.g., via color coding) in the hybrid image to provide an additional indication of the location of the scar tissue 33, or to otherwise reveal the state of the tissue in the region of the catheter's delivery portion.

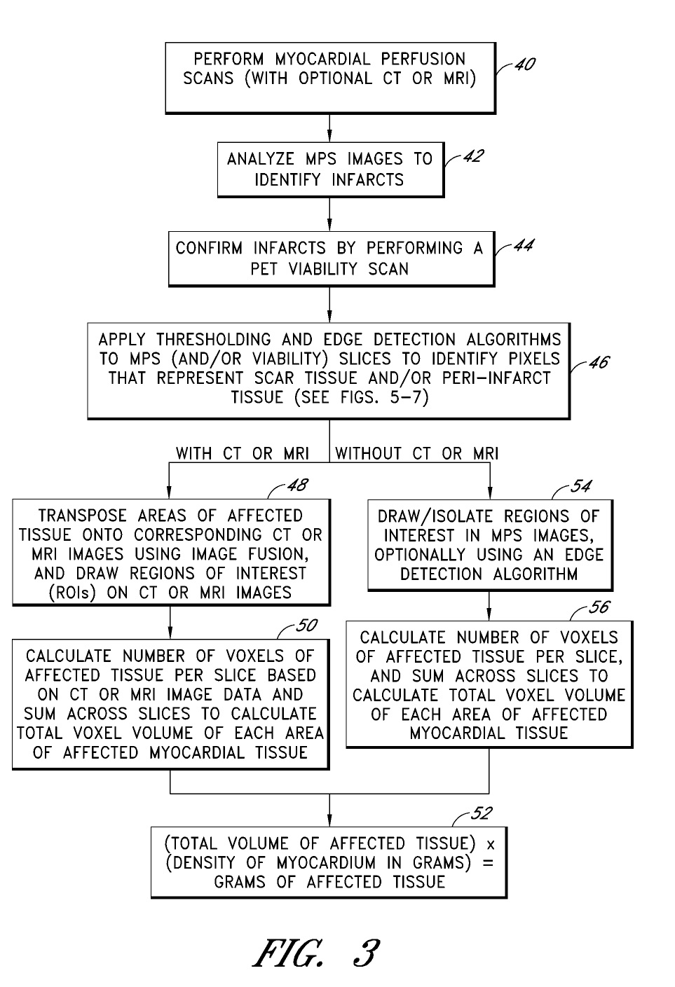

### II. GENERATION AND ANALYSIS OF NON-INVASIVE IMAGES (FIG. 3)

FIG. 3 illustrates specific examples of how non-invasive images can be generated and used to identify/quantify infarcts in steps A and B of FIG. 1. As will be apparent, numerous variations are possible. For example, although specific types of imaging studies (e.g., PET) are mentioned in these examples, other types of imaging studies may be used. The various image processing and calculation tasks depicted in FIG. 3 and described below may be performed by a computer system via execution of the image construction and analysis application 24 (FIG. 2).

As depicted in block 40 of FIG. 3, myocardial perfusion scans (MPS) of the patient are initially performed (typically using PET or SPECT) using an appropriate radioactive perfusion agent such as PET 82-Rb, 13N-ammonia, or 201-Thallium. A CT or MRI scan may also be performed (optionally using a PET/CT, PET/MRI. SPECT/CT or SPECT/MRI scanner used for the MPS scans) so that associated anatomic information is also captured. The purpose of the myocardial perfusion scans is to locate areas of scar tissue by estimating amounts of blood flow to the heart. The myocardial perfusion scans are preferably generated both at rest and under stress (either actual or drug induced), and the results are then compared. One example of a set of parameters that may be used to perform the myocardial perfusion scans using a PET scanner is provided in Table 1. The images are reconstructed by software into at least short axis (SA) images, although vertical long axis (VLA) and/or horizontal long axis (HLA) images may additionally or alternatively be used.

TABLE 1
Example parameters for PET

Fasting overnight
10-20 mCi (typical) (370-740 MBq)
Imaging acquisition Static Standard
Start time: 1.5-3 min after end of infusion
Duration: 5-15 min
Pixel size (reconstructed) 2-3 mm (Optional with 4 mm or higher)
Attenuation correction measured attenuation correction: immediately
before scan or Measured attenuation correction5
Reconstruction method iterative expectation maximization (eg, OSEM 2
order/26 subsets) heavy z axis filtering
Electrocardiographic gating of myocardium

As depicted in block 42, the MPS images are then analyzed manually and/or by computer to assess the state of the imaged organ tissue. For example, the MPS images may be analyzed to identify areas of the myocardium in which the blood flow is significantly reduced both at rest and under stress. These areas represent likely scar tissue (dead tissue or infarcts), and are the target areas for injecting stem cells and/or other therapy. Although not depicted in FIG. 3, the MPS images may also be used to identify areas of peri-infarct tissue, and/or other types of affected myocardial tissue. As with infarcts, peri-infarct tissue may benefit from the introduction of stem cells and/or other therapy.

As depicted in block 44, a PET viability study may also be conducted to confirm the infarcts identified from the MPS images. (The PET viability study can, of course, be performed either before or after the myocardial perfusion scans 40, and can be performed using the same scanner as used for MPS.) One example of a set of parameters that may be used for the PET viability study is shown in Table 2. In one embodiment, myocardial tissue is treated as scar tissue if and only if the following three conditions are met: (1) no radioactive uptake (blood flow) in the heart in the at-rest MPS scan, and (2) no radioactive uptake (blood flow) in the exact same area of the heart in the under-stress MPS scan, and (3) no uptake of FDG on FDG PET viability scan. This determination may be performed manually, or may be automated by a machine. Although depicted in FIG. 2 as a separate step, the MPS scans and PET viability scans may be analyzed concurrently and collectively.

TABLE 2
Example parameters for PET viability study

Dose 5-15 mCi (185-555 MBq) FDG after insulin manipulation
Image start time 20-60 min after injection
Image duration 10-30 min (depending on count rate and dose)
Acquisition modes 2D (3D Optional)
Static count acquisition with dynamic optional
Pixel size (reconstructed) 2-3 mm
Attenuation correction Measured attenuation correction: Simultaneous or
immediately with CT or External transmission source
Reconstruction method FBP or iterative expectation maximization (eg,
OSEM with 2 order/26subset, heavy z axis filtering
Electrocardiographic gating of myocardium

As depicted in block 46, once a determination is made that scar tissue is present, thresholding and/or edge detection algorithms may be applied to the MPS and/or viability scan images (or a combined or merged version of these two types of images) to identify the infarct boundaries. (These boundaries may alternatively be identified after fusing the MPS and/or viability scan images with CT or MRI images, such that anatomic data is considered in boundary identification.) This analysis may be performed separately on each tomography slice from the cardiac apex to the base of the heart. One example of how this analysis can be performed is provided below in a separate section.

The left hand branch in FIG. 3 depicts the steps that may be performed to measure the volume and mass of each infarct when CT or MRI data is available. As mentioned above, such data may be available if, for example, the MPS scans are generated using a PET/CT, PET/MRI, SPECT/CT or SPECT/MRI scanner, although a separate CT or MRI scanner may be used. As depicted in block 48, the infarct boundaries identified in the preceding step 46 are transposed onto corresponding CT or MRI slices/images using image fusion. (As mentioned above, the boundaries may alternatively be formed based on an analysis of MPS and/or viability scan images as fused with corresponding CT or MRI images.) These boundaries define regions of interest (ROIs) that represent scar tissue.

The combined use of nuclear (e.g., PET or SPECT) images and anatomic images (e.g., CT or MRI) enables the regions of interest, and particularly the infarct and/or peri-infarct boundaries, to ultimately be determined with a greater degree of accuracy than is possible with nuclear images alone. One reason is because the CT or MRI images, unlike the nuclear images, depict the anatomy of the heart. Thus, for example, CT or MRI images can be used to identify the wall boundaries of the left ventricle, and to ensure that the regions of interest do not extend outside such wall boundaries. Another reason is that the spatial resolution for CT and MRI (currently about 0.5 mm) is significantly better than the spatial resolution for nuclear imaging (currently about 10 to 15 mm). Further, where CT images are used, the CT images can be analyzed to detect tissue density changes characteristic of boundaries between infracted and normal tissue; this density changes can be used to confirm or refine infarct boundaries determined from the nuclear image data. As discussed below, the anatomic images are also useful for later superimposing nuclear image data onto live fluoroscopy images during a catheterization procedure.

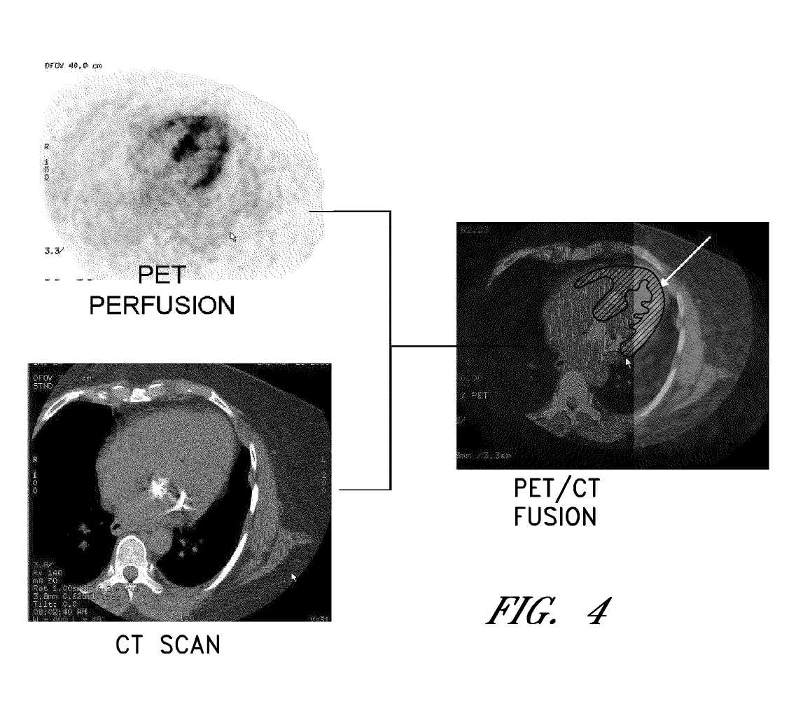

FIG. 4 illustrates the fusion of a PET perfusion (MPS) slice with a CT slice to generate a fused image showing scar tissue superimposed on a CT image of the heart. The white arrow in the fused view shows the general location of the scar tissue. Although not visible in the black and white reproduction, the areas of scar tissue are shown in the fused PET/CT view in a distinct color. (Cross hatching has been added in FIG. 4 to show the location of the color-coded representation of the scar tissue.) The fused image may be generated using software commonly provided on PET/CT scanners or with a separate software package. Distinct colors or other visual markers may also be used to show other tissue classifications determined from the nuclear scan data; for example, one or more colors may be used to show ischemic, peri-infarct, and/or or ischemic/peri-infarct tissue.

In block 50 of FIG. 3, the number of voxels of scar tissue is calculated for each ROI of each slice based on the CT or MRI image data. This involves converting pixels into voxels based on the area of each pixel and its depth (typically 3 millimeters). Because CT and MRI images include anatomic information not present in the PET or SPECT scans, the use of CT or MRI (or another appropriate anatomic imaging technology) for this purpose increases the accuracy of this volume calculation in comparison to the use of nuclear images alone. For each infarct, the voxel counts are then summed across all slices to calculate a total voxel count or volume of the infarct. A similar process may be used, if desired, to calculate the volume of any identified peri-infarct region(s).

As illustrated in blocks 54 and 56 of FIG. 3, the process for determining the regions of interest and their voxel volumes is similar if no CT or MRI images are used, but the calculations are based solely on the MPS and/or PET viability scan images.

As depicted in block 52 of FIG. 3, the total voxel volume of each infarct is then multiplied by a constant representing the density of the myocardium (approximately 1.05 grams/cm³ for scar tissue) to determine the mass (e.g., number of grams) of scar tissue in the infarct. The mass value may then be used to determine the quantity of stem cells or other therapy to inject into the infarct, and the number of injections. (As mentioned below, the doses may alternatively be determined based solely on the calculated infarct volume, without explicitly calculating infarct mass.) As mentioned above, the application software may also select (and ultimately display) target injection locations. In one embodiment, the injection locations are selected to be separated from each other by at least 1 cm. The task of selecting the injection locations may involve executing an appropriate algorithm for distributing points substantially uniformly over an irregular surface.

An area of scar tissue will frequently contain some percentage (e.g. 10 to 40%) of living cells. Thus, one possible variation to the process shown in FIG. 3 is to estimate the extent to which each ROI contains dead myocardial tissue. This may be accomplished by, for example, calculating the average pixel intensity within each region of interest relative to an appropriate reference. The results of this analysis may be incorporated into the calculation of the quantity of stem cells or other therapy to inject. Further, during the catheterization procedure, the infarcts may be displayed using color coding, with each color representing a different range or degree of damage (e.g., color 1=10 to 20% living, color 2=20 to 23% living, etc.).

### III. CLASSIFICATION AND BOUNDARY DETECTION OF AFFECTED TISSUE (FIGS. 5-8)

FIGS. 5-8 illustrate one example of a process that may be used to identify the boundaries of myocardial infarct and peri-infarct tissue using fused nuclear and anatomic images. This process generally involves (1) applying one or more thresholds to nuclear scan data to identify infarct and/or peri-infarct regions, and (2) generating a fused image in which these regions are depicted in respective colors in a corresponding anatomic or fused nuclear/anatomic image. In the illustrated examples, the analysis is performed using short axis (SA) views of the heart; however, other views, such as long axis (LA) views, may additionally or alternatively be used.

As depicted by block 60 of FIG. 5, an anatomic scan (typically CT or MRI) is initially fused with a nuclear scan (such as a PET perfusion scan) or set of nuclear scans. As mentioned above, these two types of scans may, but need not, be generated using an integrated PET/CT or PET/MRI scanner. The task of fusing the anatomic and nuclear image data may alternatively be performed after the nuclear image data has been used to identify (or preliminarily identify) the infarct and/or peri-infarct regions (i.e., after blocks 62 and 64 in FIG. 5). During the fusing process, the nuclear image data may be appropriately stretched or morphed to correspond to associated anatomic markers in the anatomic images. This may be accomplished by morphing both types of images onto the same identical map of pixels in ED space, as is known in the art.

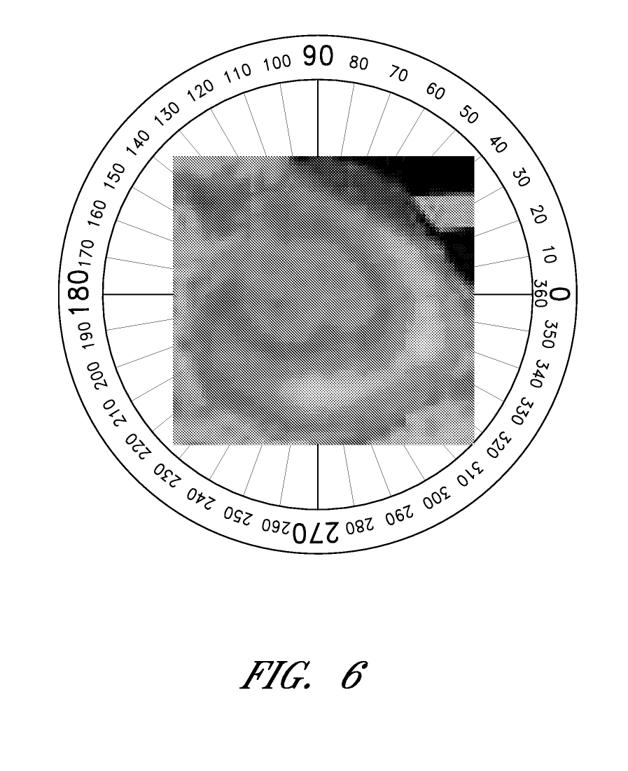

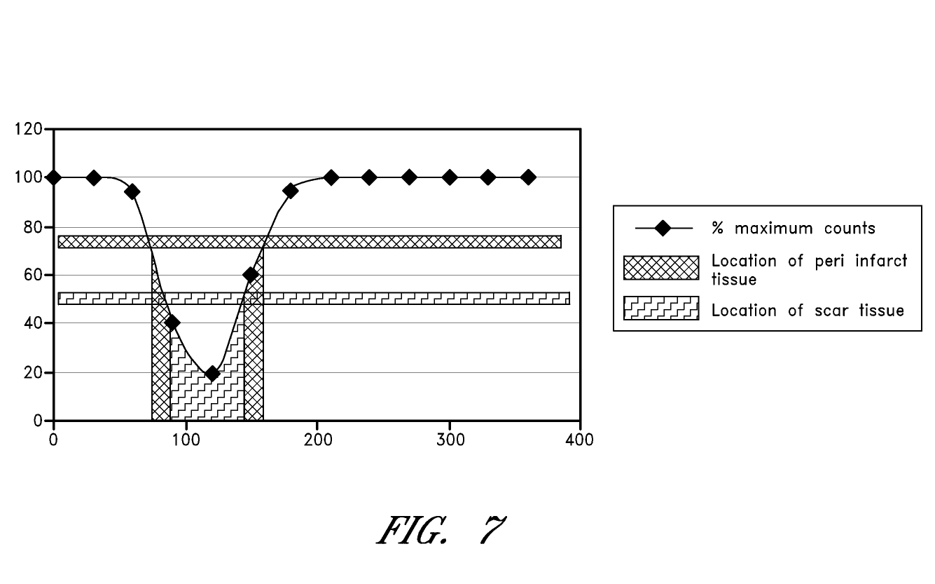

In blocks 62 and 64, each SA view or slice of the left ventricle is processed using sector analysis and threshold methods to identify and classify the regions of interest. This process is illustrated in FIGS. 6 and 7 for an example SA view generated from fused nuclear and anatomic images. As shown in FIG. 6, the fused SA view is effectively divided into angular sectors of equal size, such as 1-degree or 2-degree sectors. The nuclear scan data is then used to determine the average radioactivity level (as represented by pixel count or pixel intensity) of each sector. As is known in the art, the pixel count (i.e., counts per pixel) in a nuclear image generally represents the relative uptake of radioactivity from the tracer substance in the area corresponding to the pixel.

FIG. 7 shows a plot of average radioactivity level (pixel count) versus angular position, and illustrates how thresholds may be used to classify sectors and the pixels in such sectors. The horizontal axis in FIG. 7 goes from zero to 360 degrees, and represents the angular position along the grid of FIG. 6. Each diamond-shaped point in FIG. 7 represents the average pixel count of a respective angular sector or groups of consecutive sectors, expressed as a percentage of the maximum across all sectors. In this particular example, two thresholds are used: 75% and 50%. Sectors whose “% of maximum” value falls below 50 are classified as scar tissue. Sectors whose “% of maximum” value falls between 50 and 75 are classified as peri-infarct tissue. Sectors whose “% of maximum” value falls above 75 are classified as normal tissue. In this particular example, the tissue falling from about 90 to 150 degrees is classified as infarct, and the tissue from about 80 to 90 degrees and about 150 to 160 degrees is classified as peri-infarct. The remaining tissue is classified as normal.

The specific threshold values shown in FIG. 7 are merely illustrative, and can be varied to adjust the sensitivity of the classification process. In addition, a greater or lesser number of thresholds and associated classifications may be used. For example, a single threshold can be used, in which case each sector is classified as representing either normal tissue or an infarct. Further, three or more thresholds may be used, resulting in four or more classifications. Further, in some embodiments, different types of nuclear scan data may be used for different classifications (e.g., ischemic tissue, hibernating tissue, etc.).

As depicted by block 66 in FIG. 5, an appropriate edge detection algorithm may also be applied to the nuclear scan data to identify ventricular wall boundaries, and/or to refine the boundaries between the infarct versus peri-infarct versus normal tissue. In one embodiment, this involves using double derivatives to analyze the rate of radioactive change from pixel to pixel, and to identify the associated inflection points. This may be accomplished using the methods described in Dominique Delbeke et. al, “Estimation of Left Ventricular Mass and Infarct Size from Nitrogen-13-Ammonia PET Images Based on Pathological Examination of Explanted Human Hearts,” in The Journal of Nuclear Medicine, Vol. 4, No. 5, May 1993, pp. 826-833.

As discussed above, the anatomic image data may also be considered in identifying or refining the boundaries. For example, the anatomic images may be used to more precisely identify ventricular wall boundaries, and to identify or adjust the infarct (or peri-infarct) boundaries accordingly. As another example, CT data reflective of tissue density changes may be used to more accurately identify the boundaries between infarct (or peri-infarct) and normal tissue.

In block 68 of FIG. 5, the results of blocks 62-66 are used to generate a modified fused image in which color coding is used to reveal the tissue classifications and their boundaries along the left ventricular wall. One example of this process is shown in FIG. 8. The left hand image in FIG. 8 is a fused nuclear/anatomic image before thresholds have been used to classify particular sectors. Black lines have been added to show the location of the colored region that represents the nuclear scan image of the left ventricular wall. The image on the right in FIG. 8 shows the classifications (infarct, peri-infarct, and normal in this example) via color coding. The three colors in the original image (each representing a respective tissue classification) have been replaced with respective line patterns in this patent drawing. These three patterns correspond to those shown in FIG. 7.

As will be apparent, other approaches can be used to identify the peri-infarcts regions. In general, peri-infarct regions tend to be areas that demonstrate low or absent uptake on perfusion imaging but show FDG (radioactive glucose) uptake, indicating metabolic viability. Thus, one approach is to initially identify ischemic tissue, and to then determine whether it is adjacent to scar tissue. Ischemic tissue may be detected by, for example, identifying pixels or sectors falling in the 25-50% of maximum range on stress but not at rest.

All of the steps shown in FIG. 5 may be automated via software. Some steps may be performed by different machines or systems than others; for example, the image fusion task 60 may be performed via software executed on a PET/CT or PET/MRI scanner, while the subsequent tasks 62-66 may be performed by a separate computer system.

### IV. CALCULATION OF INFARCT/PERI-INFARCT VOLUME AND MASS

With further reference to FIG. 8, the volume of the infarct and peri-infarct regions can be calculated by multiplying the number of pixels in each such region by the area of each pixel (typically 3 mm×3 mm), and by multiplying by the depth of each pixel (typically 4 mm). For example, if the infarct (scar tissue) region in FIG. 8 has ten 3 mm×3 mm pixels, each of which has a depth of 4 mm, then the total area of scar tissue is 10 pixels×3 mm×3 mm=90 mm², and the total volume is 90 mm²×4 mm=3.6 cm³. If the infarct spans multiple slices, this volume calculation can be summed with the infarct volume calculations from the other slice(s) to obtain the total volume of the infarct. The volume of each peri-infarct region can be calculated in the same manner. Because the infarct and/or peri-infarct boundaries are preferably determined using both nuclear and anatomic image data (as described above), the volumes of the associated regions can be determined with a high degree of accuracy.

Once the volume of an infarct or peri-infarct region is known, its mass can be calculated by multiplying by the tissue density. The density of viable myocardial tissue is approximately 1.092 grams/cc, and the density for myocardial scar tissue is approximately 1.05 grams/cc. Thus, a density value falling in the range of 1.05 to 1.092 may be used, with the precise value depending on the region's classification (e.g., infarct versus peri-infarct).

### V. THERAPY DOSE CALCULATIONS

As mentioned above, the mass calculations can be used to more accurately determine the appropriate quantity or dose of therapy to inject into the affected area(s). The therapy may, for example, include the introduction of stem cells, genes/DNA, a pharmaceutical composition, and/or protein into the affected area. The type and quantity of therapy may depend on the classification and location of the affected tissue (e.g., infarct, peri-infarct, ischemic, hibernating, etc.).

For example, for stem cell therapy applied to an infarct, an approximately 1-to-1 replacement ratio may be used, such that approximately one stem cell is injected for every cell of dead myocardial tissue. The optimum replacement ratio can be determined over time through experimentation. Typically, one gram of myocardium contains approximately 20 million cells. In one embodiment, the number of stem cells to inject into an infarct is calculated as: (grams of scar tissue)×(20,000,000 cells/gram)×K, where K is a scaling factor that accounts for the optimum replacement ratio and the presence of living cells. The value of K may, for example, be in the range of 0.5 to 1.5.

In practice, because the density of myocardial tissue is relatively constant regardless of its state (e.g., infarct versus peri-infarct), the dose can be determined based solely on the calculated volume of the affected tissue, without explicitly calculating the mass of such tissue. For example, once the volume of an infarct is known, the volume can simply be multiplied by a constant—without first converting volume to mass—to determine the dose of the therapeutic substance to be injected into the infarct. Thus, where this document refers to the use of mass calculations to determine therapy doses, it should be understood that an explicit mass calculation may not be necessary.

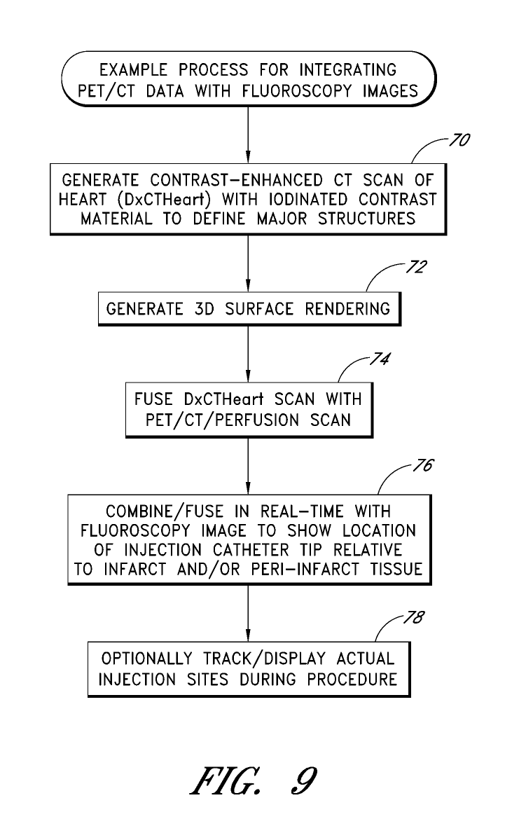

### VI. INTEGRATION OF NON-INVASIVE IMAGES INTO THE CATHETERIZATION LAB (FIGS. 9-11)

As explained above, some of the non-invasive/static image data generated in step A of FIG. 1 may, in some embodiments, be incorporated into the catheterization lab. FIG. 8 illustrates one particular example of how this may be done. In this particular example, a PET/CT based view of scar tissue is superimposed, via image registration or fusing, onto a live fluoroscopy image of the heart. As will be apparent (and as discussed above), numerous variations are possible. For example, catheter location information derived from fluoroscopy images (and/or location sensors) may be incorporated in real time into a three-dimensional PET/CT, PET/MRI, or other static image of the heart.

As depicted by block 70 of FIG. 9, a contrast-enhanced CT scan of the heart is generated using an iodinated contrast material. This CT scan, referred to herein as DxCTHeart, is separate from the PET/CT scan used for scar detection. The purpose of the DxCTHeart scan is to generate images that clearly show the major structures of the heart, such that these structures can be used as references for subsequently fusing PET/CT images with fluoroscopy images. The major structures shown in the DxCTHeart images preferably include the chambers, the pulmonary vessels, the superior and inferior vena cava, and the cardiac vessels.

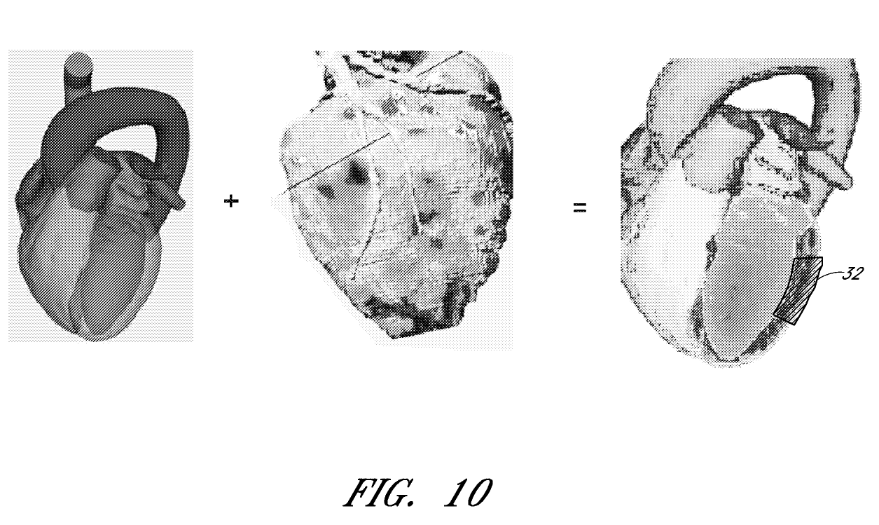

As depicted by block 72 of FIG. 9, 3D surface rendering software of the type commonly provided on CT scanners is used to generate (1) a 3D surface rendering of the heart based on the DxCTHeart image data, and (2) a 3D surface rendering of the heart based on the PET/CT image data. These two 3D surface renderings are depicted in FIG. 10, with the DxCTHeart rendering shown on the left. In block 74 of FIG. 9, the two 3D surface renderings are fused to generate a fused image that reveals the location of scar tissue within the left ventricular wall. The fused image is shown on the right in FIG. 10, with cross hatching added in place of the original color coding to show the location of the scar tissue 32. In this particular example, only the scar tissue, and not the peri-infarct tissue, is shown; however, peri-infarct tissue may be shown in a similar manner.

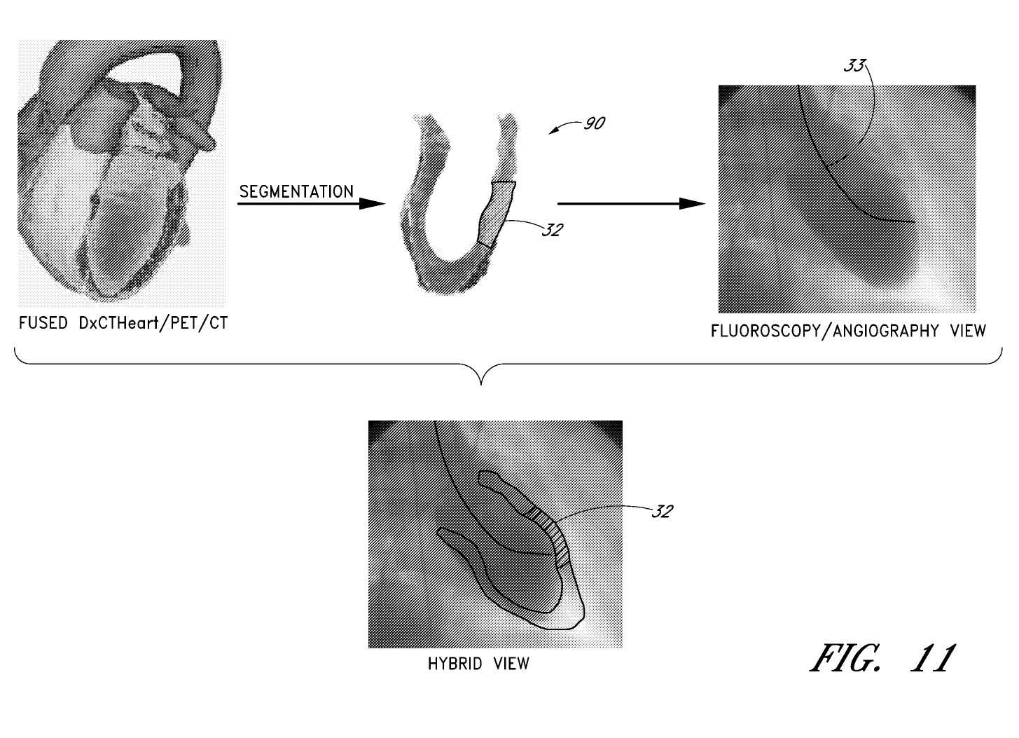

As indicated in block 76 of FIG. 9, the fused DxCTHeart/PET/CT 3D rendering is imported onto a real time navigation system used in the catheterization lab, and is fused with live fluoroscope images during the subsequent procedure. This may be accomplished using fusion methods similar to those described in Kriatselis et al., supra. FIG. 11 illustrates one example of this process. In this example, segmentation is initially used to separate out a color-coded representation 90 of the left ventricle, with the scar tissue shown in a unique color (represented by cross hatching in FIG. 11). This color-coded representation of the left ventricle is then superimposed in real time by image integration software onto a fluoroscopy image of the heart to produce a hybrid view. This hybrid view illustrates the location of the catheter, including the injection needle, relative to the scar tissue.

Although the hybrid view in FIG. 11 is two-dimensional, a 3D view may alternatively be generated by fusing a color-coded representation of the scar tissue with a 3D fluoroscopy view. Another option for effectively showing the catheter location in three dimensions is to use two perpendicularly oriented fluoroscopy cameras to generate views of the heart, and to fuse respective representations of the scar tissue with each of these fluoroscopy images.

As depicted in block 78 of FIG. 9 and discussed above, the real time navigation system may also track and display the actual injection locations during the subsequent procedure. Further, the system may automatically update or generate new target injection locations (which may be visually depicted as colored dots on the hybrid view) based on the actual injection locations.

As will be apparent, numerous additional variations to the process shown in FIGS. 9-11 are possible. For example, one or more other types of non-invasive images that show the scar tissue (e.g., PET/MRI, SPECT/CT, SPECT/MRI, CT alone, or MRI alone) may alternatively be fused with the fluoroscopy image to generate the hybrid view. Further, the process depicted in FIGS. 9-11 can also be applied to other organs, including those listed above.

### VII. OTHER APPLICATIONS

As will be apparent, the medical imaging and medical treatment methods disclosed herein can be used to analyze and treat a variety of different types of affected organ tissue, including but not limited to the following: (1) both malignant and benign tumors of solid organs, (2) infections of the chest, lungs, liver, pancreas, kidneys and bladder, brain and spinal cord, muscles and bones, (3) trauma, including injury from blunt trauma, penetrating trauma, falls, accidents, burns, electrical shock, chemicals, and inhalants, (4) inflammatory and immune conditions that affect multiple organ systems, such as lupus, arthritis, diabetes, and pulmonary-renal syndromes, (5) congenital and developmental conditions that result in loss of function in organs and limbs for which regeneration of tissue would at least partially if not completely restore function, (6) degenerative conditions that affect the brain (such as dementia like Alzheimer's, frontal temporal dementia, lewy body dementia, subcortical dementias, vascular dementia), neuromuscular syndromes like Parkinson's disease, Lou Gehrig's disease, and Muscular Dystrophy, and (7) vascular insufficiency and inflammatory vascular diseases like myocardial ischemia, infarction, hibernating myocardium, stunned myocardium, myocarditis, congestive heart failure, atherosclerosis, stroke, and ischemia and infarction of major organ systems.

Further, in addition to the organs mentioned above, the disclosed methods can be applied to organ systems such as, but not limited to, the following: (1) the central nervous system, which includes the brain and the spinal cord; (2) the sensory system, which includes the organs of the five senses with major emphasis on sight and sound, (3) the muscular skeletal system, (4) the cardiovascular system, including the heart and blood vessels, (5) the pulmonary system, which includes the lungs and heart, (6) the GI system, from the mouth to the anus with organs of digestion including the stomach, small intestines, colon, gall bladder, pancreas, and liver, (7) the genital urinary system, which includes the kidneys, bladder, and prostate, (8) the endocrine system, which includes the pituitary gland, thyroid gland, parathyroid gland, adrenal glands, and pancreas, and (9) the immune system, which includes the liver, spleen, bone marrow, and thymus.

### VIII. CONCLUSION

The various image generation and processing tasks disclosed herein may be fully automated in code modules executed by a computer system. The computer system may, in some embodiments, include multiple distinct physical computers or machines that communicate over a network. The code modules may be stored in any type of types of physical computer storage (magnetic disk drives, solid state RAM and ROM devices, optical disks, etc.).

As will be apparent, many of the implementation details set forth above can be omitted or varied. In addition, some of the features disclosed herein may be implemented without others; for example, the disclosed processes for calculating the volume or mass of damaged organ tissue may be implemented without the disclosed processes for incorporating static image data into the catheterization lab (and vice versa). Accordingly, nothing in the foregoing description is intended to imply that any particular feature or detail is essential to any of the inventions disclosed herein. The inventive subject matter is defined by the appended claims.

What is claimed is:

1. A method of determining a quantity of therapy to deliver to the myocardium of a patient, the method comprising:
obtaining nuclear image data representing at least one nuclear medicine scan of the heart of a patient;
obtaining anatomic image data representing an anatomic scan of the heart of the patient, said anatomic scan being distinct from the nuclear scan;
identifying boundaries of a region of affected myocardial tissue of the heart at least partly by programmatically analyzing the nuclear image data in combination with the anatomic image data with a computer system, said affected myocardial tissue including infarct tissue and/or peri-infarct tissue;
calculating a volume of the region of affected myocardial tissue using the identified boundaries; and
calculating a quantity of therapy to deliver to the region of affected myocardial tissue based at least partly on the calculated volume.

2. The method of claim 1, further comprising, by execution of software by a computer, superimposing a visual representation of the boundaries of said region onto a fluoroscopy image substantially in real time to enable a physician to guide a percutaneously inserted injection catheter to said region.

3. The method of claim 1, further comprising, by execution of software by a computer, combining a visual representation of said region with live image data generated during a cardiac catheterization procedure to generate a view which shows, substantially in real time, a location of a delivery portion of an injection catheter relative to said region, said visual representation of the region generated at least partly from the nuclear image data.

4. The method of claim 1, wherein the nuclear image data represents a cardiac positron emission tomography (PET) scan.

5. The method of claim 4, wherein the anatomic image data represents a computerized tomography (CT) scan or a magnetic resonance imaging (MRI) scan of the heart.

6. The method of claim 1, wherein identifying the boundaries comprises analyzing a fused representation of the nuclear and anatomic image data.

7. The method of claim 1, wherein identifying the boundaries comprises determining initial boundaries based on the nuclear image data, and adjusting the initial boundaries based on the anatomic image data.

8. The method of claim 1, wherein identifying the boundaries comprises using tissue density information included in a CT image, in combination with pixel count information included in the nuclear image data, to identify a boundary of the region.

9. The method of claim 1, wherein calculating the quantity of therapy comprises calculating a quantity of stem cells to inject into the region.

10. The method of claim 9, wherein calculating the quantity of stem cells comprises using the volume calculation to calculate the mass of the region of affected myocardial tissue, and using the calculated mass to calculate said quantity of stem cells.

11. The method of claim 9, further comprising injecting the calculated quantity of stem cells into the region using a percutaneously inserted injection catheter.

12. The method of claim 11, wherein the injection catheter includes a voltage sensor, and the method further comprises using the voltage sensor to take voltage measurements along a myocardial wall to confirm a presence of an infarct.

13. The method of claim 1, further comprising, after said quantity of therapy has been delivered to the affected myocardial tissue:
repeating said steps of obtaining nuclear and atomic image data, identifying boundaries, and calculating a volume, to thereby generate a second volume calculation; and
using said second volume calculation to assess a degree to which the therapy caused a reduction in a quantity of affected myocardial tissue.

14. A computer system programmed to perform the method of claim 1, said computer system comprising one or more physical computers.

15. Physical computer storage which stores executable code that instructs a computer system to perform the method of claim 1.

16. A method of determining a quantity of therapy to apply to affected organ tissue of a patient, the method comprising:
obtaining nuclear image data representing at least one nuclear medicine scan of an organ of the patient;
obtaining anatomic image data representing an anatomic scan of the organ of the patient, said anatomic scan being distinct from the nuclear scan;
identifying boundaries of a region of affected tissue of said organ at least partly by programmatically analyzing the nuclear image data in combination with the anatomic image data with a computer system such that the boundaries are determined based on both (1) tissue state information included in the nuclear scan image data, and (2) anatomic structural information included in the anatomic image data;
calculating a volume of the region of affected tissue using the identified boundaries; and
calculating a quantity of therapy to inject into the region of affected tissue based at least partly on the calculated volume.

17. The method of claim 16, further comprising, by execution of software by a computer, superimposing a visual representation of the boundaries onto a fluoroscopy image substantially in real time to enable a physician to guide a percutaneously inserted injection catheter to said region.

18. The method of claim 16, wherein nuclear image data represents a positron emission tomography (PET) scan of the organ.

19. The method of claim 18, wherein the anatomic image data represents a computerized tomography (CT) scan or a magnetic resonance imaging (MRI) scan of the organ.

20. The method of claim 16, wherein the region of affected tissue comprises an infarct.

21. The method of claim 16, wherein the region of affected tissue comprises a myocardial infarct.

22. The method of claim 16, wherein the region of affected tissue comprises a region of peri-infarct tissue.

23. The method of claim 16, wherein identifying the boundaries comprises analyzing a fused image data generated by fusing the nuclear image data with the anatomic image data.

24. The method of claim 16, wherein identifying the boundaries comprises determining initial boundaries based on the nuclear image data, and adjusting the initial boundaries based on the anatomic image data.

25. The method of claim 16, wherein identifying the boundaries comprises using tissue density information included in a CT image, in combination with pixel count information included in the nuclear image data, to identify a boundary of an infarct.

26. The method of claim 16, wherein calculating the quantity of therapy comprises calculating a quantity of stem cells to inject into the region of affected tissue.

27. The method of claim 26, wherein calculating the quantity of stem cells comprises using the volume calculation to calculate the mass of the region of affected tissue, and using the calculated mass to calculate said quantity of stem cells.

28. The method of claim 26, further comprising injecting the calculated quantity of stem cells into the region of affected tissue using a percutaneously inserted injection catheter.

29. The method of claim 28, wherein the injection catheter includes a voltage sensor, and the method further comprises using the voltage sensor to take voltage measurements along a wall of the organ to check for infarct tissue.

30. A computer system programmed to perform the method of claim 16, said computer system comprising one or more physical computers.

31. Physical computer storage which stores executable code that instructs a computer system to perform the method of claim 16.
